(19) United States
(12) Patent Application Publication
(10) Pub. No.: US 2022/0328843 A1
(43) Pub. Date: Oct. 13, 2022

(54) ELECTROCONDUCTIVE COMPOSITE STRUCTURE AND METHOD FOR PRODUCING SAME

(71) Applicant: Murata Manufacturing Co., Ltd., Nagaokakyo-shi (JP)

(72) Inventor: Masanori ABE, Nagaokakyo-shi (JP)

(21) Appl. No.: 17/835,700

(22) Filed: Jun. 8, 2022

Related U.S. Application Data
(63) Continuation of application No. PCT/JP2020/045508, filed on Dec. 7, 2020.

(30) Foreign Application Priority Data
Dec. 25, 2019 (JP) 2019-233667

(51) Int. Cl. H01M 4/66; H01M 4/04; H01M 4/46; H01M 10/0525; H01G 11/30; H01G 11/26; H01G 11/86

(52) U.S. Cl. CPC H01M 4/667; H01M 4/661; H01M 4/0416; H01M 4/0404; H01M 4/463; H01M 10/0525; H01G 11/30; H01G 11/26; H01G 11/86; A61B 5/263

(57) ABSTRACT

A conductive composite structure having a metal substrate and a conductive film on a surface of the metal substrate, the conductive film including a layered material of one or plural layers; the one or plural layers being a layer body represented by MₘXₙ, where M is at least one metal of Group 3, 4, 5, 6 or 7; X is a carbon atom, a nitrogen atom, or a combination thereof; n is not less than 1 and not more than 4; and m is more than n but not more than 5, and a modifier or terminal T exists on a surface of the layer body; and a residue derived from an organic compound having a hydroxyl group, a carbonyl group, or a combination thereof and having 2 to 8 carbon atoms, is bonded to each of the surface of the metal substrate and a surface of the layer body.

### CROSS REFERENCE TO RELATED APPLICATIONS

The present application is a continuation of International application No. PCT/JP2020/045508, filed Dec. 7, 2020, which claims priority to Japanese Patent Application No. 2019-233667, filed Dec. 25, 2019, the entire contents of each of which are incorporated herein by reference.

### FIELD OF THE INVENTION

The present invention relates to a conductive (electroconductive) composite structure, more specifically, to a conductive composite structure comprising a metal substrate (base material) and a conductive film provided on a surface of the metal substrate, and a production method thereof.

### BACKGROUND OF THE INVENTION

In recent years, MXene has been attracting attention as a new material having conductivity. MXene is a type of so-called two-dimensional material, and as will be described later, is a layered material in the form of one or plural layers.

It is known that MXene can be utilized as an electrode active material of an electrochemical capacitor (particularly a pseudo capacitor) or a lithium ion battery (see, for example, Patent Literature 1). An electrode utilizing MXene as an electrode active material can be produced as a conductive film containing a mixture containing MXene and a binder, and in some cases, can be produced as a conductive film containing only MXene. An electrode utilizing MXene as an electrode active material can also be produced by forming such a conductive film on the surface of a current collector composed of a metal substrate. In more detail, a slurry containing MXene as an electrode active material, a binder, and an organic solvent is prepared, and the slurry is coated onto the current collector, dried, and pressed to be fixed. (See paragraphs 0020, 0026, 0042 etc. of Patent Literature 1.)

Patent Literature 1: JP 2016-63171 A

### SUMMARY OF THE INVENTION

A conventionally known conductive film containing only MXene has the drawbacks that it is difficult to maintain the form of the film by MXene alone, a crack can occur when the film is bent, and resilience to being bent is low. In comparison, a conductive film containing a mixture containing MXene and a binder is improved in resilience to being bent, but the surface and/or interlayers of MXene can be hindered by the binder. So, it is difficult to obtain sufficiently high resilience to being bent while the electrical characteristics of MXene itself are sufficiently utilized. Furthermore, when a conductive film containing only MXene or a conductive film containing a mixture containing MXene and a binder is formed on the surface of a current collector composed of a metal substrate according to a conventionally known method, there is the drawback that the bonding between the conductive film and the metal substrate is not sufficient and the conductive film is easily peeled off.

An object of the present invention is to provide a conductive composite structure comprising a metal substrate and a conductive film provided on a surface of the metal substrate, wherein the conductive film contains MXene and has high resilience to being bent, and bonding strength between the conductive film and the metal substrate is high. A further object of the present invention is to provide a method for producing such a conductive composite structure.

According to one aspect of the present invention, a conductive composite structure is provided, which comprises a metal substrate and a conductive film on a surface of the metal substrate.

The conductive film comprises a layered material comprising one or plural layers, the one or plural layers comprising a layer body represented by:

MₘXₙ

wherein M is at least one metal of Group 3, 4, 5, 6, or 7, X is a carbon atom, a nitrogen atom, or a combination thereof, n is not less than 1 and not more than 4, and m is more than n but not more than 5, and

a modifier or terminal T existing on a surface of the layer body, wherein T is a hydroxyl group, an oxygen atom, or a combination thereof.

A residue derived from an organic compound, having a hydroxyl group, a carbonyl group, or a combination thereof and having not less than 2 and not more than 8 carbons atoms, is bonded to each of the surface of the metal substrate and the surface of the layer body.

According to another aspect of the present invention, a method for producing a conductive composite structure comprises:

(a) preparing a dispersion liquid in which a layered material is dispersed in a liquid medium containing an organic compound having a hydroxyl group, a carbonyl group, or a combination thereof and having not less than 2 and not more than 8 carbons atoms, the layered material comprising one or plural layers,

the one or plural layers comprising
a layer body represented by:

MₘXₙ
wherein M is at least one metal of Group 3, 4, 5, 6, or 7, X is a carbon atom, a nitrogen atom, or a combination thereof, n is not less than 1 and not more than 4, and m is more than n but not more than 5, and
a modifier or terminal T existing on a surface of the layer body, wherein T is a hydroxyl group, an oxygen atom, or a combination thereof;

(b) applying the dispersion liquid to a surface of a metal substrate; and

(c) subjecting the metal substrate to which the dispersion liquid has been applied to a heat treatment so as to form the conductive composite structure comprising the metal substrate and a conductive film on the surface of the metal substrate.

According to the conductive composite structure of the present invention, a conductive composite structure comprising a metal substrate and a conductive film on a surface of the metal substrate is provided, in which the conductive film contains a predetermined layered material (also referred to as “MXene” in the present description), and a residue derived from an organic compound, having a hydroxyl group, a carbonyl group, or a combination thereof and having not less than 2 and not more than 8 carbon atoms, is bonded to each of the surface of the metal substrate and the surface of the layer body of the layered material, whereby the conductive composite structure has high resilience to being bent and high bonding strength between the conductive film and the metal substrate. In addition, according to the method for producing a conductive composite structure of the present invention, a conductive composite structure can be produced, in which: a dispersion liquid in which MXene is dispersed in a liquid medium containing an organic compound, having a hydroxyl group, a carbonyl group, or a combination thereof and having not less than 2 and not more than 8 carbon atoms, is used; the dispersion liquid is applied to a surface of a metal substrate; and the metal substrate is subjected to a heat treatment, whereby the conductive composite structure has high resilience to being bent and high bonding strength between a conductive film and the metal substrate.

### BRIEF DESCRIPTION OF THE DRAWINGS

FIG. 1 is a schematic cross-sectional view showing a conductive composite structure according to one embodiment of the present invention.

FIG. 2 is a schematic cross-sectional view showing MXene that is a layered material that can be utilized in the conductive composite structure according to the one embodiment of the present invention.

FIGS. 3(a) to 3(c) are photographs showing evaluation results of a conductive composite structure produced in Example 1 of the present invention, in which FIG. 3(a) shows a result of a shaft center winding test, FIG. 3(b) shows a result of an acetone immersion test, and FIG. 3(c) shows a result of a tape peeling test.

FIGS. 4(a) to 4(c) are photographs showing evaluation results of a conductive composite structure produced in Example 2 of the present invention, in which FIG. 4(a) shows a result of a shaft center winding test, FIG. 4(b) shows a result of an acetone immersion test, and FIG. 4(c) shows a result of a tape peeling test.

FIGS. 5(a) to 5(c) are photographs showing evaluation results of a conductive composite structure produced in Example 3 of the present invention, in which FIG. 5(a) shows a result of a shaft center winding test, FIG. 5(b) shows a result of an acetone immersion test, and FIG. 5(c) shows a result of a tape peeling test.

FIGS. 6(a) to 6(c) are photographs showing evaluation results of a conductive composite structure produced in Example 4 of the present invention, in which FIG. 6(a) shows a result of a shaft center winding test, FIG. 6(b) shows a result of an acetone immersion test, and FIG. 6(c) shows a result of a tape peeling test.

FIGS. 7(a) to 7(c) are photographs showing evaluation results of a conductive composite structure produced in Comparative Example 1, in which FIG. 7(a) shows a result of a shaft center winding test, FIG. 7(b) shows a result of an acetone immersion test, and FIG. 7(c) shows a result of a tape peeling test.

### DETAILED DESCRIPTION OF THE PREFERRED EMBODIMENTS

Hereinafter, a conductive composite structure according to one embodiment of the present invention will be described in detail through a production method thereof, but the present invention is not limited to such an embodiment.

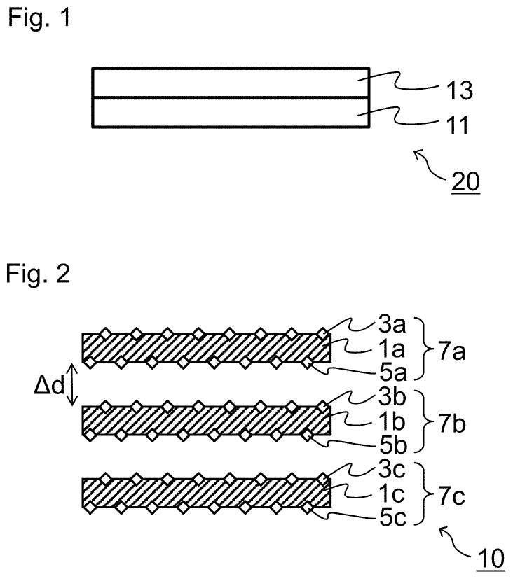

With reference to FIG. 1, a conductive composite structure 20 of the present embodiment comprises a metal substrate 11 and a conductive film 13 provided on a surface of the metal substrate 11.

A method for producing the conductive composite structure 20 of the present embodiment comprises:

(a) preparing a dispersion liquid in which a predetermined layered material is dispersed in a liquid medium containing an organic compound having a hydroxyl group, a carbonyl group, or a combination thereof (in other words, a hydroxyl group and/or an oxygen atom) and having not less than 2 and not more than 8 carbon atoms;

(b) applying the dispersion liquid to a surface of a metal substrate; and

(c) subjecting the metal substrate to which the dispersion liquid has been applied to a heat treatment.

Step (a)

First, the predetermined layered material is prepared. The predetermined layered material that can be used in the present embodiment is MXene, which is defined as follows:

MXene is a layered material comprising one or plural layers, the one or plural layers comprising a layer body represented by a formula below:

MₘXₙ

wherein M is at least one metal of Group 3, 4, 5, 6, or 7, and can comprise at least one selected from the group consisting of so-called early transition metals, for example, Sc, Ti, Zr, Hf, V, Nb, Ta, Cr, Mo, and Mn,

X is a carbon atom, a nitrogen atom, or a combination thereof, n is not less than 1 and not more than 4, and

m is more than n but not more than 5

(the layer body can have a crystal lattice in which each X is located in the octahedral array of M), and

a modifier or terminal T existing on a surface of the layer body (in more detail, on at least one of both surfaces, facing each other, of the layer body), wherein T is a hydroxyl group, an oxygen atom, or a combination thereof (in other words, a hydroxyl group and/or an oxygen atom, and can further optionally be a fluorine atom and/or a hydrogen atom) (the layered material can be understood as a layered compound and also represented by “MₘXₙTₛ,” wherein s is any number and traditionally x may be used instead of s). Typically, n can be 1, 2, 3, or 4, but is not limited thereto.

In the present embodiment, MXene has a hydroxyl group and/or an oxygen atom as the modifier or terminal T, and preferably has both a hydroxyl group and an oxygen atom. A hydroxyl group and/or an oxygen atom existing as the modifier or terminal T in MXene contribute to the reaction to be described later.

In the above formula of MXene, M is preferably at least one selected from the group consisting of Ti, Zr, Hf, V, Nb, Ta, Cr, and Mo.

Such MXene can be synthesized by selectively etching (removing and optionally layer-separating) A atoms (and optionally parts of M atoms) from a MAX phase. The MAX phase is represented by the formula below:

MₘAXₙ

wherein M, X, n, and m are as described above, A is at least one element of Group 12, 13, 14, 15, or 16, normally an element of Group A, typically of Group IIIA and Group IVA, in more detail can comprise at least one selected from the group consisting of Al, Ga, In, Tl, Si, Ge, Sn, Pb, P, As, S, and Cd, and is preferably Al; and has a crystal structure in which a layer composed of A atoms is located between the two layers represented by MₘXₙ (can have a crystal lattice in which each X is located in the octahedral array of M). When typically m=n+1, but not limited thereto, the MAX phase includes repeating units in which each one layer of X atoms is disposed in between adjacent layers of n+1 layers of M atoms (these are also collectively referred to as an “MₘXₙ layer”), and a layer of A atoms (“A atom layer”) is disposed as a layer next to the (n+1)th layer of M atoms. The A atom layer (and optionally a part of the M atoms) is removed by selectively etching (removing and optionally layer-separating) the A atoms (and optionally a part of the M atoms) from the MAX phase. By selectively etching (removing and optionally layer-separating) the A atoms (and optionally a part of the M atoms) from the MAX phase, the A atomic layer (and optionally a part of the M atoms) is removed, and hydroxyl groups and/or oxygen atoms (further optionally fluorine atoms and/or hydrogen atoms, etc.) existing in an etching solution (usually, but not limited to, an aqueous solution of a fluorine-containing acid is used) are modified on the exposed surface of the MₘXₙ layer, thereby terminating the surface. The etching can be carried out using an etching liquid containing F⁻, and a method using, for example, a mixed liquid of lithium fluoride and hydrochloric acid, a method using hydrofluoric acid, or the like may be used. Then, the layer separation of MXene (delamination, separating multilayer MXene into single-layer MXene and/or few-layer MXene) may be appropriately promoted by any suitable post-treatment (e.g., ultrasonic treatment, handshake, automatic shaker, or the like). Since the shear force of an ultrasonic treatment is too large so that the MXene particles can be destroyed, it is desirable to apply an appropriate shear force by handshake, an automatic shaker or the like, when it is desired to obtain a two-dimensional MXene (preferably single-layer MXene and/or few-layer MXene) having a larger aspect ratio.

MXenes whose above formula MₘXₙ is expressed as below are known:

Sc₂C, Ti₂C, Ti₂N, Zr₂C, Zr₂N, Hf₂C, Hf₂N, V₂C, V₂N, Nb₂C, Ta₂C, Cr₂C, Cr₂N, Mo₂C, Mo_{1.3}C, Cr_{1.3}C, (Ti,V)₂C, (Ti,Nb)₂C, W₂C, W_{1.3}C, Mo₂N, Nb_{1.3}C, Mo_{1.3}Y_{0.6}C (in the above formulae, “1.3” and “0.6” mean about 1.3 (=4/3) and about 0.6 (=2/3), respectively),

Ti₃C₂, Ti₃N₂, Ti₃(CN), Zr₃C₂, (Ti,V)₃C₂, (Ti₂Nb)C₂, (Ti₂Ta)C₂, (Ti₂Mn)C₂, Hf₃C₂, (Hf₂V)C₂, (Hf₂Mn)C₂, (V₂Ti)C₂, (Cr₂Ti)C₂, (Cr₂V)C₂, (Cr₂Nb)C₂, (Cr₂Ta)C₂, (Mo₂Sc)C₂, (Mo₂Ti)C₂, (Mo₂Zr)C₂, (Mo₂Hf)C₂, (Mo₂V)C₂, (Mo₂Nb)C₂, (Mo₂Ta)C₂, (W₂Ti)C₂, (W₂Zr)C₂, (W₂Hf)C₂,

Ti₄N₃, V₄C₃, Nb₄C₃, Ta₄C₃, (Ti,Nb)₄C₃, (Nb,Zr)₄C₃, (Ti₂Nb₂)C₃, (Ti₂Ta₂)C₃, (V₂Ti₂)C₃, (V₂Nb₂)C₃, (V₂Ta₂)C₃, (Nb₂Ta₂)C₃, (Cr₂Ti₂)C₃, (Cr₂V₂)C₃, (Cr₂Nb₂)C₃, (Cr₂Ta₂)C₃, (Mo₂Ti₂)C₃, (Mo₂Zr₂)C₃, (Mo₂Hf₂)C₃, (Mo₂V₂)C₃, (Mo₂Nb₂)C₃, (Mo₂Ta₂)C₃, (W₂Ti₂)C₃, (W₂Zr₂)C₃, and (W₂Hf₂)C₃.

Typically in the above formula, M can be titanium or vanadium and X can be a carbon atom or a nitrogen atom. For example, the MAX phase is Ti₃AlC₂, and MXene is Ti₃C₂Tₛ.

It is noted, in the present invention, MXene may contain remaining A atoms at a relatively small amount, for example, at 10% by mass or less with respect to the original amount of A atoms. The remaining amount of A atoms can be preferably 8% by mass or less, and more preferably 6% by mass or less. However, even if the remaining amount of A atoms exceeds 10% by mass, there can be no problem depending on the use and conditions of use of the conductive composite structure.

As schematically shown in FIG. 2, the MXene 10 synthesized in this way can be a layered material containing one or plural MXene layers 7a, 7b, and 7c (in the figure, three layers are exemplarily shown, but not limited thereto). More specifically, the MXene layers 7a, 7b, and 7c have layer bodies (MₘXₙ layers) 1a, 1b, and 1c represented by MₘXₙ, and modifiers or terminals T 3a, 5a, 3b, 5b, 3c, and 5c existing on the surfaces of the layer bodies 1a, 1b, and 1c (in more detail, on at least one of both surfaces, facing each other, of each layer). Therefore, the MXene layers 7a, 7b, and 7c are also represented by “MₘXₙTₛ,” wherein s is any number. MXene 10 may be: one that exists as one layer obtained by such MXene layers being separated individually (single-layer structure, so-called single-layer MXene); a laminate composed of a plurality of MXene layers stacked apart from each other (multilayer structure, so-called multilayer MXene); or a mixture thereof. MXene can be particles (which can also be referred to as powders or flakes) as an aggregation composed of the single-layer MXene and/or the multilayer MXene. In the case of the multilayer MXene, two adjacent MXene layers (e.g., 7a and 7b, 7b and 7c) may not necessarily be completely separated from each other, and may be partially in contact with each other.

Although not limiting the present embodiment, the thickness of each layer of MXene (which corresponds to the MXene layer 7a, 7b, or 7c) is, for example, not less than 0.8 nm and not more than 5 nm, and particularly not less than 0.8 nm and not more than 3 nm (which can vary mainly depending on the number of M atom layers contained in each layer), and the maximum dimension in a plane (two-dimensional sheet plane) parallel to the layer is, for example, not less than 0.1 μm and not more than 200 μm, and particularly not less than 1 μm and not more than 40 μm.

In a case where MXene is a laminate (multilayer MXene), the inter-layer distance (or gap dimension, denoted as Ad in FIG. 2) in the individual laminate is, for example, not less than 0.8 nm and not more than 10 nm, particularly not less than 0.8 nm and not more than 5 nm, and more particularly about 1 nm. The maximum dimension in a plane (two-dimensional sheet plane) perpendicular to the stacking direction is, for example, not less than 0.1 μm and not more than 100 μm, and particularly not less than 1 μm and not more than 20 μm.

In a case where MXene is a laminate (multilayer MXene), the total number of layers in the individual laminate may be 2 or more but is, for example, not less than 50 and not more than 100,000, and particularly not less than 1,000 and not more than 20,000. The thickness in the stacking direction is, for example, not less than 0.1 μm and not more than 200 μm, and particularly not less than 1 μm and not more than 40 μm.

In a case where MXene is a laminate (multilayer MXene), MXene having a small number of layers may be used. The term “small number of layers” means, for example, that the number of stacked layers of MXene is 6 or less. The thickness, in the stacking direction, of the multilayer MXene having a small number of layers is preferably 10 nm or less. In the present description, this “multilayer MXene having a small number of layers” (multilayer MXene in a narrow sense) is also referred to as “few-layer MXene.”

In the present embodiment, most of MXene 10 may be particles (which can also be referred to as nanosheets) composed of single-layer MXene 10a and/or few-layer MXene. In other words, the proportion of particles (single-layer MXene and/or few-layer MXene) having a thickness, in the stacking direction, of 10 nm or less in the entire MXene particles can be 50% by volume or more.

It should be noted that these dimensions described above are determined as number average dimensions (e.g., number average of at least 40) based on photographs of a scanning electron microscope (SEM), a transmission electron microscope (TEM), or an atomic force microscope (AFM), or as distances in the real space calculated from the positions on the reciprocal lattice space of the (002) plane measured by an X-ray diffraction (XRD) method.

Separately, a liquid medium containing an organic compound having a hydroxyl group and/or a carbonyl group and having not less than 2 and not more than 8 carbon atoms (hereinafter, also simply referred to as a “reactive organic compound”) is prepared.

The reactive organic compound has a hydroxyl group and/or a carbonyl group, and typically can have any one of a hydroxyl group and a carbonyl group. The hydroxyl group and/or carbonyl group of the reactive organic compound contribute to the reaction to be described later.

The reactive organic compound has not less than 2 and not more than 8, preferably not less than 3 and not more than 7 carbon atoms. When the number of carbon atoms is not less than 2, preferably not less than 3, aggregation of MXene in the dispersion liquid (before the heat treatment to be described later) can be prevented. When the number of carbon atoms is not more than 8, preferably not more than 7 and when MXene is a laminate (multilayer MXene), the reactive organic compound can appropriately penetrate between layers of the laminate.

The reactive organic compound can be an alcohol or ketone having not less than 2 and not more than 8 carbon atoms. In more detail, the reactive organic compound can be at least one selected from the group consisting of isopropyl alcohol, N-methylpyrrolidone, and methyl ethyl ketone, or can be any one thereof or a mixture of any two or more thereof.

The liquid medium preferably consists of the reactive organic compound, but may contain, in addition to the reactive organic compound, other organic compounds in a relatively small amount (e.g., 30% by mass or less, preferably 20% by mass or less on the whole basis). The liquid medium may contain a very small amount of water (e.g., 30% by mass or less, preferably 20% by mass or less on the whole basis), depending on the reactive organic compound to be used.

A dispersion liquid (which can also be referred to as a suspension or a slurry) in which the above MXene is dispersed in such a liquid medium is prepared. A dispersion method is not particularly limited, but typically stirring (share mixer, pot mill, etc.), ultrasonic treatment, shaking, or the like can be used.

In this dispersion liquid, the reactive organic compound can be in contact with the surface of MXene, and when MXene is a laminate (multilayer MXene), the reactive organic compound can be in contact with the outermost surface of the laminate and can penetrate between layers of the laminate.

The content ratio of MXene in the dispersion liquid is not particularly limited, and can be, for example, 20% to 95% by mass.

Step (b)

First, the metal substrate 11 is prepared. The metal substrate 11 may be a conductive member whose base is one type or two or more types of metals. The “conductive member whose base is a metal” means a member in which the content of the metal in the metal substrate 11 (when two or more types of metals are contained, the total content of the metals) is 80% by weight or more, for example, 90% by weight or more, and preferably 95% by weight or more, the member being conductive as a whole. Examples of a conductive substance other than the metal that can form the metal substrate 11 include carbon.

The metal substrate 11 has a hydroxyl group, an oxygen atom, or a combination thereof (In other words, a hydroxyl group and/or an oxygen atom) on its outermost surface, and preferably has both a hydroxyl group and an oxygen atom. The hydroxyl group and/or oxygen atom existing on the outermost surface of the metal substrate 11 contribute to the reaction to be described later.

The metal substrate 11 can typically have a sheet-like form. The “sheet-like” refers to, as generally understood, a shape having two planes facing each other and having a relatively small distance (thickness) between the planes, and can also be referred to as a film, a foil, or the like in addition to a sheet. However, the metal substrate 11 is not limited thereto, and may have any suitable form.

It should be noted that even when the metal substrate 11 is a metal member, not all of the metal substrate are strictly composed of only metal atoms. The hydroxyl group and/or oxygen atom existing on the outermost surface of the metal substrate 11 may be ones existing on the outermost surface of a metal oxide film (in more detail, which is a film of an amorphous metal oxide, and is a so-called passivation film) existing in a surface portion of the metal substrate 11.

The metal substrate 11 may be an aluminum substrate, a copper substrate, or a stainless steel substrate (in other words, a conductive member whose base is aluminum, copper, or stainless steel), and in more detail, may be an aluminum member, a copper member, or a stainless steel member. The aluminum member has a film of alumina (which can be amorphous) formed in its surface portion, and the outermost surface of the alumina film has a hydroxyl group and/or an oxygen atom, usually both a hydroxyl group and an oxygen atom. The copper member has a film of copper oxide (which can be amorphous) formed in its surface portion, and the outermost surface of the copper oxide film has a hydroxyl group and/or an oxygen atom, usually both a hydroxyl group and an oxygen atom. The stainless steel is steel having a carbon content of 1.2% by mass or less and a chromium content of 10.5% by mass or more, and can optionally contain an additive metal such as nickel. The stainless steel may be, for example, SUS 304, SUS 316, SUS 430, or the like. The stainless steel member has a film of iron oxide and chromium oxide (which can be amorphous) formed in its surface portion, and the outermost surface of the iron oxide-chromium oxide film has a hydroxyl group and/or an oxygen atom, usually both a hydroxyl group and an oxygen atom.

However, the metal substrate 11 (e.g., an aluminum substrate, a copper substrate, or a stainless steel substrate) may have a conductive substance other than metal, as described above. Typically, a carbon-coating layer may be formed on a metal member (e.g., an aluminum member, a copper member, or a stainless steel member). Unless subjected to a special treatment such as a hydrophobic treatment, carbon has, on its outermost surface, a hydroxyl group and/or an oxygen atom, usually both a hydroxyl group and an oxygen atom.

The dimension of the metal substrate 11 is not particularly limited, and can be appropriately selected depending on the use of the conductive composite structure 20. The thickness of the metal substrate 11 is preferably one at which the substrate can be bent, but this is not essential in use where high resilience to being bent is not required.

Then, the dispersion liquid prepared in the step (a) is applied to (in more detail, coated onto) the surface of the metal substrate 11. An application method is not particularly limited, and for example, blade coat, knife coat, bar coat, screen printing, slit coat, die coat, roll coat, dip coat, spray coat, spin coat, or the like can be utilized.

The thickness of the dispersion liquid applied to the surface of the metal substrate 11 can vary depending on the composition of the dispersion liquid, the thickness desired for the conductive film 13, and the like.

Step (c)

The metal substrate 11 to which the dispersion liquid has been applied in the step (b) is subjected to a heat treatment.

By the heat treatment, the reactive organic compound can be reacted with the metal substrate 11 and can be reacted with MXene 10. In more detail, the following reaction can proceed.

When the reactive organic compound has a hydroxyl group, the hydroxyl group of the reactive organic compound can react with the oxygen atom existing on the outermost surface of the metal substrate 11 to form a bond therebetween. In addition, the hydroxyl groups of the reactive organic compound can react with the oxygen atoms existing as the modifiers or terminals T on the surfaces of the layer bodies 1a, 1b, and 1c of MXene 10 to form bonds therebetween. More specifically, these reactions can be, but are not limited to, reactions each forming a hydrogen bond between the hydrogen atom of the hydroxyl group of the reactive organic compound and the oxygen atom of the metal substrate 11/MXene 10. The reactions may involve, for example, a reaction in which a hydrogen atom is eliminated from the reactive organic compound, and the like.

When the reactive organic compound has a carbonyl group, the carbonyl group of the reactive organic compound can react with the hydroxyl group existing on the outermost surface of the metal substrate 11 to form a bond therebetween. In addition, the carbonyl groups of the reactive organic compound can react with the hydroxyl groups existing as the modifiers or terminals T existing on the surfaces of the layer bodies 1a, 1b, and 1c of MXene 10 to form bonds therebetween. More specifically, these reactions can be, but are not limited to, reactions each forming a hydrogen bond between the oxygen atom of the carbonyl group of the reactive organic compound and the hydrogen atom of the metal substrate 11/MXene 10. The reactions may involve, for example, a reaction in which a hydrogen atom is eliminated from the reactive organic compound, and the like.

Although the present invention is not bound by any theory, the reaction mechanism can be understood as schematically shown below. In the following, a case where isopropyl alcohol reacts with MXene and a case where isopropyl alcohol reacts with a copper member are illustratively shown as a case where the reactive organic compound has a hydroxyl group, while a case where N-methylpyrrolidone reacts with MXene, a case where N-methylpyrrolidone reacts with an aluminum member, and a case where methyl ethyl ketone reacts with an aluminum member are illustratively shown as a case where the reactive organic compound has a carbonyl group, but other cases can be similarly understood.

Case Where the Reactive Organic Compound has a Hydroxyl Group

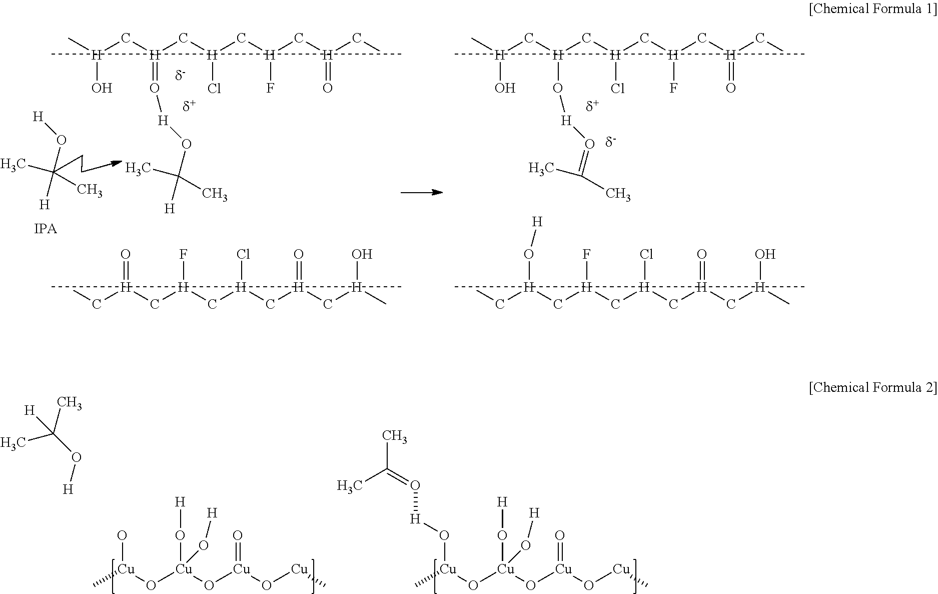

Case Where the Reactive Organic Compound has a Carbonyl Group

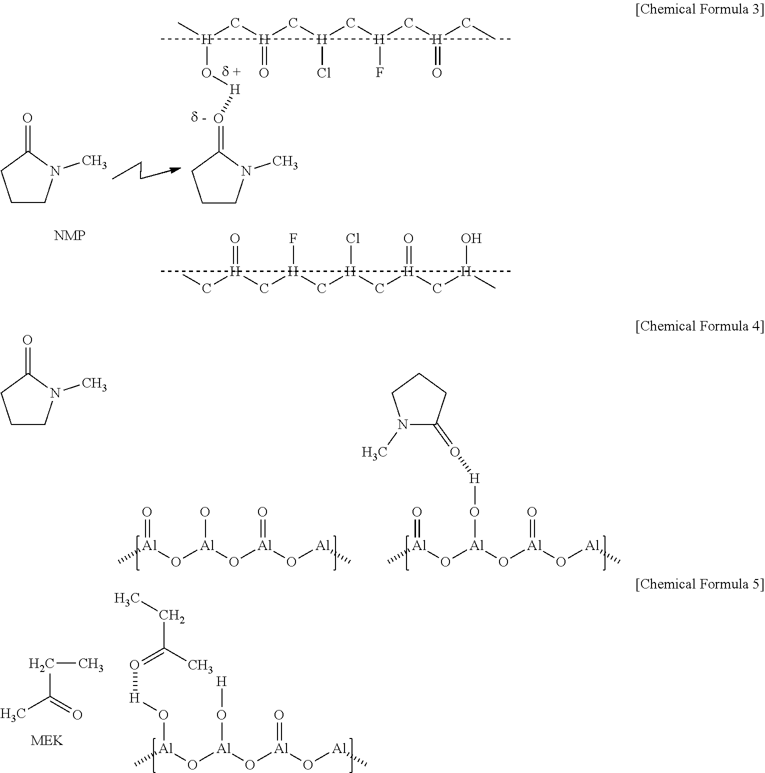

In addition, an unreacted reactive organic compound can be evaporated and removed by the heat treatment.

During the heat treatment, the reaction of the reactive organic compound and the evaporation and removal proceed, so that MXene (regardless of whether it is a single-layer MXene or a multilayer MXene) can be tightly aggregated.

Heat treatment conditions can vary depending on the reactive organic compound to be used. A heat treatment temperature can be, for example, not lower than 70° C. and not higher than 200° C., and preferably not lower than 80° C. and not higher than 180° C. A heat treatment time can be appropriately set, and can be, for example, not shorter than 0.5 hours and not longer than 24 hours. A heat treatment atmosphere can be a reduced pressure (or vacuum) atmosphere, an air atmosphere, a nitrogen atmosphere, or the like.

As a result of the heat treatment, the conductive film 13 derived from the dispersion liquid is formed on the surface of the metal substrate 11, whereby the conductive composite structure 20 of the present embodiment is produced (see FIG. 1). Note that in the method for producing the conductive composite structure 20 of the present embodiment, any suitable post-step may be performed after the heat treatment of the step (c). Such a post-step can be a step of cutting (e.g., punching) the conductive composite structure 20 into a desired shape and/or a step of pressing the conductive composite structure 20, and the like.

In the conductive composite structure 20 of the present embodiment, a residue derived from the reactive organic compound is bonded to the surface of the metal substrate 11, and a residue derived from the reactive organic compound is bonded to the surfaces of the layer bodies 1a, 1b, and 1c of MXene (in other words, the surface and/or interlayers of MXene). The “residue derived from the reactive organic compound” means a residual substance of the reactive organic compound after the heat treatment. For example, as compared with the chemical formula of the original reactive organic compound, the “residue derived from the reactive organic compound” may be what is represented by the same chemical formula except for the hydrogen bond forming part, or may be what is represented by a chemical formula changed by the elimination of a hydrogen atom, or the like. It should be noted that in the conductive composite structure 20 of the present embodiment, unreacted hydroxyl groups and/or oxygen atoms remain as the modifiers or terminals T on the surfaces of the layer bodies 1a, 1b, and 1c of MXene.

In addition, in the conductive composite structure 20 of the present embodiment, the unreacted reactive organic compound is evaporated and removed, so that the conductive film 13 can be understood as a dry film.

Furthermore, in the conductive composite structure 20 of the present embodiment, the conductive film 13 contains MXene and does not contain a binder (in a binderless manner), and is provided on the surface of the metal substrate 11. For MXene and the metal substrate in the conductive composite structure 20, the description in the production method of the present embodiment can be similarly applied unless otherwise specified.

According to the present embodiment, the conductive composite structure 20, having high resilience to being bent (particularly high flexibility) and high bonding strength between the conductive film 11 and the metal substrate 13 (in particular, chemical stability to an organic solvent, peel strength, etc.), can be produced. In more detail, a crack hardly occurs in the conductive film 13 even when the conductive composite structure 20 is bent, and the conductive film 13 is hardly peeled off from the metal substrate 11 even when the conductive composite structure 20 is immersed in an organic solvent or subjected to a tape peeling test. The present invention is not bound by any theory, but it can be understood that the reason why high resilience to being bent is obtained in the conductive composite structure 20 is that due to the existence of the residue derived from the reactive organic compound on the surfaces and/or interlayers of MXene, the density of MXene of the conductive film 13 becomes higher than a conventionally known conductive film consisting of only MXene, and the adhesion strength between MXene (single-layer MXene and/or multilayer MXene) becomes high, so that the strength, shapability, and flexibility of the conductive film itself are improved (even in the case of being binderless). In addition, it can be understood that the reason why high bonding strength is obtained between the conductive film 13 and the metal substrate 11 is that due to the existence of the residue derived from the reactive organic compound on both the surface of MXene and the surface of the metal substrate, the residue derived from the reactive organic compound functions like an adhesive between the conductive film 13 and the metal substrate 11.

The conductive composite structure 20 of the present embodiment can be utilized in any suitable use. Since the conductive composite structure 20 of the present embodiment has a high density of MXene of the conductive film 13 and can achieve desired electrical characteristics with smaller dimensions, it can be preferably utilized in a case where miniaturization of the conductive composite structure 20 and eventual miniaturization of a product in which the conductive composite structure is incorporated are required. In addition, the conductive composite structure 20 of the present embodiment can be preferably utilized in a case where the conductive composite structure 20 is required to be resilient to being bent and stretched during use by a user of a final product in which it is incorporated and/or during a production process until it is incorporated in the final product.

Particularly preferably, the conductive composite structure 20 of the present embodiment can be used as an electrode. When the conductive composite structure 20 is used as an electrode, MXene of the conductive film 13 functions as an electrode active material (a substance that sends and receives electrons with electrolyte ions in an electrolytic solution), and the metal substrate 11 functions as a current collector.

The electrode is not particularly limited, and can be, for example, a capacitor electrode, a battery electrode, a bioelectrode, a sensor electrode, an antenna electrode, or the like. When the conductive composite structure 20 of the present embodiment is used, and even when the conductive composite structure has a smaller volume (device occupied volume), a capacitor and a battery each having a large capacity, a bioelectrode having a low impedance, and a sensor and an antenna each having a high sensitivity can be obtained.

The capacitor can be an electrochemical capacitor. The electrochemical capacitor is a capacitor utilizing capacitance developed due to a physicochemical reaction between an electrode (electrode active material) and ions (electrolyte ions) in an electrolytic solution, and can be used as a device (power storage device) that stores electrical energy. The battery can be a chemical battery that can be repeatedly charged and discharged. The battery can be, for example, a lithium ion battery, a magnesium ion battery, a lithium sulfur battery, a sodium ion battery, or the like, but it is not limited thereto. In the production processes of the capacitor and the batteries, the electrodes can be required to have flexibility to be resistant to being bent and stretched, and the electrodes can be disposed in a bent manner in the capacitor and the batteries, and in such use, the conductive composite structure 20 of the present embodiment can be suitably utilized as the electrode.

The biological electrode is an electrode for acquiring a biological signal. The bioelectrode can be, for example, an electrode for measuring EEG (electroencephalogram), ECG (electrocardiogram), EMG (electromyogram), or EIT (electrical impedance tomography), but it is not limited thereto. The bioelectrode can be used by being attached to a living body (in particular, skin), and is required to have flexibility to be resistant to being bent and stretched without being peeled off from the skin even when the skin stretches and contracts. In such use, the conductive composite structure 20 of the present embodiment can be suitably utilized as the electrode.

The sensor electrode is an electrode for detecting a target substance, state, abnormality, or the like. The sensor can be, for example, a gas sensor, a biosensor (a chemical sensor utilizing a molecular recognition mechanism of biological origin), or the like, but it is not limited thereto. When the conductive composite structure 20 of the present embodiment is utilized as the sensor electrode, a sensor electrode, having high bonding strength with a metal substrate and being entirely flexible, can be provided.

The antenna electrode is an electrode for emitting an electromagnetic wave into space and/or receiving an electromagnetic wave in space. When the conductive composite structure 20 of the present embodiment is utilized as the antenna electrode, an antenna electrode, having high bonding strength with a metal substrate and being entirely flexible, can be provided.

The conductive composite structure according to one embodiment of the present invention has been described in detail through its production method, but various modifications can be made. It should be noted that the conductive composite structure of the present invention may be produced by a different method from the production method in the above embodiment, and the method for producing the conductive composite structure of the present invention is not limited only to those that provide the conductive composite structure in the above embodiment.

### EXAMPLES

### Example 1

The present example relates to an example in which a conductive composite structure was produced by using isopropyl alcohol (IPA) as the reactive organic compound and a copper foil as the metal substrate.

Preparation of MXene Powder

Ti₃AlC₂ particles were prepared as the MAX particles by a known method. One g of the Ti₃AlC₂ particles (powder) was weighed, added to 10 mL of 9 mol/L hydrochloric acid together with 1 g of LiF, and stirred with a stirrer at 35° C. for 24 hours to obtain a solid-liquid mixture (suspension) containing a solid component derived from the Ti₃AlC₂ powder. Regarding this mixture, the operation of washing with pure water and separating and removing a supernatant liquid using a centrifuge (the remaining sediment excluding the supernatant was subjected to the washing again) was repeated about 10 times to obtain a MXene slurry obtained by adding pure water to the sediment.

The obtained MXene slurry was subjected to freeze drying, and the aggregated dry powder was crushed with a tube mill control (manufactured by IKA). As a result, Ti₃C₂Tₛ powder was obtained as the MXene powder.

Preparation of Dispersion Liquid

The MXene powder (Ti₃C₂Tₛ powder) prepared above and isopropyl alcohol (IPA), the content ratio of the MXene powder being 30% by mass (on the whole basis), were stirred with a thin-film spin system high-speed mixer (manufactured by PRIMIX Corporation, Model: 40-L). The stirring was repeated until a substantially uniform MXene-IPA dispersion liquid was obtained while the mixture was cooled to 20° C. with ice water after each stirring.

Coating of Dispersion Liquid onto Metal Substrate

The MXene-IPA dispersion liquid prepared above was coated onto the upper surface of a copper foil (manufactured by THANK METAL Co., Ltd., thickness: 10 μm) as the metal substrate by using a table coater (manufactured by TESTER SANGYO Co., Ltd., PI-1210 automatic coating device). Note that as the table coater, a variable blade (manufactured by YOSHIIMITSU SEIKI Co., Ltd., Baker Applicator YBA-3) set so as to have a gap of 127 μm was used.

Heat Treatment

The copper foil onto which the MXene-IPA dispersion liquid was coated above was subjected to a heat treatment in a vacuum (vacuum degree: 0.1 kPa) at 120° C. for 3 hours in a vacuum oven (manufactured by AS ONE Corporation, ETTAS vacuum dryer AVO-310 SB). As a result, a structure in which a MXene dry film (hereinafter, referred to as an “IPA-MXene film”) derived from the IPA dispersion liquid was formed on the upper surface of the copper foil was obtained as a conductive film.

Pressing

Thereafter, the structure obtained above was pressed with a roll press machine at a linear pressure of 0.6 kN/cm and a conveying speed of 0.3 m/min.

As a result, a conductive composite structure of Example 1 was obtained.

Evaluation

The conductive composite structure of Example 1 produced above was subjected to a shaft center winding test, an acetone immersion test, and a tape peeling test to be evaluated.

In the shaft center winding test, the conductive composite structure was wound about two turns around an aluminum round bar having a diameter of 4 mm, and while the state was maintained, the presence or absence of crack and peeling-off was visually observed.

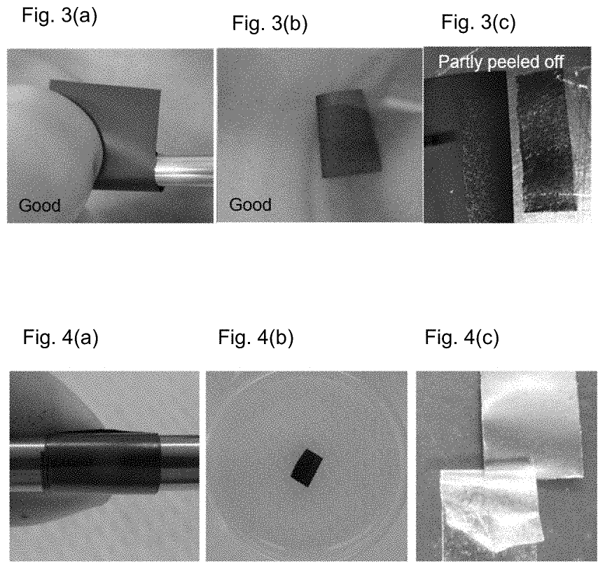

As a result, neither crack in the IPA-MXene film nor peeling-off of the IPA-MXene film from the copper foil was observed at all, as shown in FIG. 3(a).

In the acetone immersion test, the conductive composite structure was cut to obtain a test piece of 1 cm square. This test piece was immersed in acetone that is one type of organic solvent, at room temperature for 1 minute, and the presence or absence of peeling was visually observed.

As a result, the IPA-MXene film maintained its original shape, and no peeling was observed at all between the IPA-MXene film and the copper foil, as shown in FIG. 3(b).

In the tape peeling test, a cellophane adhesive tape (manufactured by NICHIBAN Co., Ltd., “CELLOTAPE” (registered trademark)) was attached to a part of the upper surface of the IPA-MXene film of the conductive composite structure. Thereafter, the tape was peeled off, and the presence or absence of peeling by the tape (transfer to the tape adhesive surface) was visually observed.

As a result, in the IPA-MXene film, only a portion of about 5% in total of the attachment region was sparsely peeled off from the copper foil by the tape, as shown in FIG. 3(c).

### Example 2

The present example relates to an example in which a conductive composite structure was produced by using isopropyl alcohol (IPA) as the reactive organic compound and an aluminum foil as the metal substrate. Note that unless otherwise specified, the same devices as in Example 1 are used, and the same production conditions and evaluation methods are applied (the same applies to the following Examples and Comparative Examples).

Preparation of MXene Powder

A MXene slurry obtained in the same way as in Example 1 was subjected to freeze drying, and the aggregated dry powder was crushed with a planetary ball mill. As a result, Ti₃C₂Tₛ powder was obtained as the MXene powder.

Preparation of Dispersion Liquid

The MXene powder (Ti₃C₂Tₛ powder), in an amount of 5.8 g, prepared above and 13.6 g of isopropyl alcohol (IPA) were placed in a glass container, and the glass container containing a mixture thereof was immersed in an ultrasonic bath filled with water and subjected to an ultrasonic treatment for 1 hour. The ultrasonic treatment was performed until a substantially uniform MXene-IPA dispersion liquid was obtained.

Coating of Dispersion Liquid onto Metal Substrate

The MXene-IPA dispersion liquid prepared above was coated onto the upper surface of an aluminum foil (manufactured by THANK METAL Co., Ltd., thickness: 10 μm) as the metal substrate by using a table coater.

Heat Treatment

The aluminum foil onto which the MXene-IPA dispersion liquid was coated above was subjected to a preliminary heat treatment (100° C., for 15 min) on a hot plate, and then subjected to a heat treatment in a vacuum (vacuum degree: 0.1 kPa) at 120° C. for 3 hours in a vacuum oven. As a result, a structure in which a MXene dry film (hereinafter, referred to as an “IPA-MXene film”) derived from the IPA dispersion liquid was formed on the upper surface of the aluminum foil was obtained as a conductive film.

Pressing

Thereafter, the structure obtained above was pressed with a roll press machine at a linear pressure of 4.4 kN/cm and a conveying speed of 0.3 m/min.

As a result, a conductive composite structure of Example 2 was obtained.

Evaluation

The conductive composite structure of Example 2 produced above was subjected to the shaft center winding test, the acetone immersion test, and the tape peeling test in the same way as in Example 1 in order for the structure to be evaluated. As a result of the shaft center winding test, neither crack in the IPA-MXene film nor peeling-off of the IPA-MXene film from the aluminum foil was observed at all, as shown in FIG. 4(a). As a result of the acetone immersion test, the IPA-MXene film maintained its original shape, and no peeling was observed at all between the IPA-MXene film and the aluminum foil, as shown in FIG. 4(b). As a result of the tape peeling test, the IPA-MXene film was not peeled off from the aluminum foil at all by the tape, as shown in FIG. 4(c). Example 2 showed the best results among Examples 1 to 4.

### Example 3

The present example relates to an example in which a conductive composite structure was produced by using N-methylpyrrolidone (NMP) as the reactive organic compound and an aluminum foil as the metal substrate.

Preparation of MXene Powder

A MXene slurry obtained in the same way as in Example 1 was subjected to freeze drying, and the aggregated dry powder was crushed with a tube mill control (manufactured by IKA). As a result, Ti₃C₂Tₛ powder was obtained as the MXene powder.

Preparation of Dispersion Liquid

The MXene powder (Ti₃C₂Tₛ powder), in an amount of 9.5 g, prepared above and 18 g of N-methylpyrrolidone (NMP) were stirred with a rotation/revolution type stirrer. During the stirring, 4 g of NMP was added and they were further stirred. The stirring was performed until a substantially uniform MXene-NMP dispersion liquid was obtained.

Coating of Dispersion Liquid onto Metal Substrate

The MXene-NMP dispersion liquid prepared above was coated onto the upper surface of an aluminum foil (manufactured by THANK METAL Co., Ltd., thickness: 10 μm) as the metal substrate by using a table coater.

Heat Treatment

The aluminum foil onto which the MXene-NMP dispersion liquid was coated above was subjected to a preliminary heat treatment (80° C., for 10 min) on a hot plate, and then subjected to a heat treatment in a vacuum (vacuum degree: 0.1 kPa) at 100° C. for 10 hours in a vacuum oven. As a result, a structure in which a MXene dry film (hereinafter, referred to as an “NMP-MXene film”) derived from the NMP dispersion liquid was formed on the upper surface of the aluminum foil was obtained as a conductive film.

Pressing

Thereafter, the structure obtained above was pressed with a roll press machine at a linear pressure of 0.6 kN/cm and a conveying speed of 0.3 m/min.

As a result, a conductive composite structure of Example 3 was obtained.

Evaluation

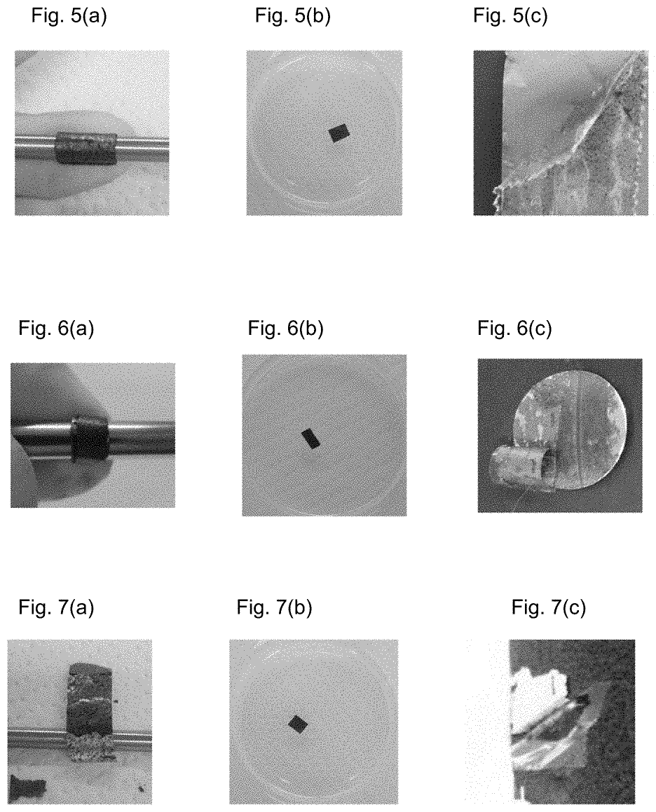

The conductive composite structure of Example 3 prepared above was subjected to the shaft center winding test, the acetone immersion test, and the tape peeling test in the same way as in Example 1 in order for the structure to be evaluated. As a result of the shaft center winding test, neither crack in the NMP-MXene film nor peeling-off of the NMP-MXene film from the aluminum foil was observed at all, as shown in FIG. 5(a). As a result of the acetone immersion test, the NMP-MXene film maintained its original shape, and no peeling was observed at all between the NMP-MXene film and the aluminum foil, as shown in FIG. 5(b). As a result of the tape peeling test, the NMP-MXene film was not peeled off from the aluminum foil at all by the tape, as shown in FIG. 5(c).

### Example 4

The present example relates to an example in which a conductive composite structure was produced by using methyl ethyl ketone (MEK) as the reactive organic compound and an aluminum foil as the metal substrate.

Preparation of MXene Powder

Ti₃C₂Tₛ powder was obtained as the MXene powder, in the same way as in Example 3.

Preparation of Dispersion Liquid

The MXene powder (Ti₃C₂Tₛ powder), in an amount of 5.8 g, prepared above and 13.5 g of methyl ethyl ketone (MEK) were stirred with a rotation/revolution type stirrer. During the stirring, 1.8 g of the MXene powder and 4.3 g of distilled water were added and they were further stirred. The stirring was performed until a substantially uniform MXene-MEK dispersion liquid was obtained.

Coating of Dispersion Liquid onto Metal Substrate

The MXene-MEK dispersion liquid prepared above was coated onto the upper surface of an aluminum foil (manufactured by THANK METAL Co., Ltd., thickness: 10 μm) as the metal substrate by using a table coater.

Heat Treatment

The aluminum foil onto which the MXene-MEK dispersion liquid was coated above was subjected to a preliminary heat treatment (80° C., for 10 min) on a hot plate, and then subjected to a heat treatment in a vacuum (vacuum degree: 0.1 kPa) at 100° C. for 10 hours in a vacuum oven. As a result, a structure in which a MXene dry film (hereinafter, referred to as a “MEK-MXene film”) derived from the MEK dispersion liquid was formed on the upper surface of the aluminum foil was obtained as a conductive film.

Pressing

Thereafter, the structure obtained above was pressed with a roll press machine at a linear pressure of 0.6 kN/cm and a conveying speed of 0.3 m/min.

As a result, a conductive composite structure of Example 4 was obtained.

Evaluation

The conductive composite structure of Example 4 prepared above was subjected to the shaft center winding test, the acetone immersion test, and the tape peeling test in the same way as in Example 1 in order for the structure to be evaluated. As a result of the shaft center winding test, neither crack in the MEK-MXene film nor peeling-off of the MEK-MXene film from the aluminum foil was observed at all, as shown in FIG. 6(a). As a result of the acetone immersion test, the MEK-MXene film maintained its original shape, and no peeling was observed at all between the MEK-MXene film and the aluminum foil, as shown in FIG. 6(b). As a result of the tape peeling test, in the MEK-MXene film, only a portion of about 20% in total of the attachment region was peeled off from the aluminum foil by the tape, as shown in FIG. 6(c).

### Comparative Example 1

The present comparative example relates to an example in which a conductive composite structure was produced by using an aluminum foil as the metal substrate without using the reactive organic compound.

Preparation of MXene Clay

Ti₃AlC₂ particles were prepared as the MAX particles by a known method. One g of the Ti₃AlC₂ particles (powder) was weighed, added to 10 mL of 9 mol/L hydrochloric acid together with 1 g of LiF, and stirred with a stirrer at 35° C. for 24 hours to obtain a solid-liquid mixture (suspension) containing a solid component derived from the Ti₃AlC₂ powder. Regarding this mixture, the operation of washing with pure water and separating and removing a supernatant liquid using a centrifuge (the remaining sediment excluding the supernatant was subjected to the washing again) was repeated about 10 times to obtain a clay-like substance (clay) as a sediment. As a result, a Ti₃C₂Tₛ-water dispersion clay was obtained as a MXene clay.

Preparation of Dispersion Liquid

The MXene clay (Ti₃C₂Tₛ-water dispersion clay), in an amount of 24.4 g, prepared above was stirred with a thin-film spin system high-speed mixer (manufactured by PRIMIX Corporation, Model: 40-L). During the stirring, 8.4 g of the MXene powder (Ti₃C₂Tₛ powder) prepared in the same way as in Example 1 was added and they were further stirred to obtain an MXene-water dispersion liquid. The stirring was performed until a substantially uniform MXene-water dispersion liquid was obtained.

Coating of Dispersion Liquid onto Metal Substrate, Heat Treatment, and Pressing

Coating of the dispersion liquid onto the aluminum foil, a heat treatment, and pressing were performed in the same way as in Example 3 except that the MXene-water dispersion liquid prepared above was used instead of the MXene-MEK dispersion liquid.

As a result, a conductive composite structure of Comparative Example 1 was obtained.

Evaluation

The conductive composite structure of Comparative Example 1 prepared above was subjected to the shaft center winding test, the acetone immersion test, and the peeling test in the same way as in Example 1 in order for the structure to be evaluated. As a result of the shaft center winding test, it was found that a crack occurred in the water-MXene film, the water-MXene film was peeled off from the aluminum foil, and the water-MXene film was weak against being bent, as shown in FIG. 7(a). As a result of the acetone immersion test, no peeling was observed between the water-MXene film and the aluminum foil, as shown in FIG. 7(b). As a result of the tape peeling test, in the water-MXene film, a large area portion exceeding about 70% of the attachment region was peeled off from the aluminum foil by the tape, as shown in FIG. 7(c), so that the bonding strength between the water-MXene film and the aluminum foil was low.

### Comparative Example 2

The present comparative example relates to an example in which dimethyl ether (DME) was used as the organic compound and an aluminum foil was used as the metal substrate.

Preparation of MXene Powder

Ti₃C₂Tₛ powder was obtained as the MXene powder, in the same way as in Example 3.

Preparation of Dispersion Liquid

The MXene powder (Ti₃C₂Tₛ powder), in an amount of 5.8 g, prepared above and 13.5 g of dimethyl ether (DME) were stirred with a rotation/revolution type stirrer. During the stirring, 1.8 g of the MXene powder and 4.3 g of distilled water were added and they were further stirred. In these mixtures, a lump product was formed, a substantially uniform dispersion liquid could not be prepared, and a MXene-DME mixture containing the lump product was obtained.

Coating of Dispersion Liquid onto Metal Substrate

An attempt was made to coat the MXene-DME mixture obtained above onto the upper surface of an aluminum foil (manufactured by THANK METAL Co., Ltd., thickness: 10 μm) as the metal substrate by using a table coater. However, it was difficult to coat the MXene-DME mixture. In addition, the mixture was peeled off from the aluminum foil after being coated.

Therefore, in Comparative Example 2, the tests were stopped when the MXene-DME mixture was peeled off from (did not adhere to) the aluminum foil, and a conductive composite structure could not be produced.

The conductive composite structure of the present invention can be utilized in any suitable application, can be preferably utilized in applications where miniaturization and/or resilience to being bent and stretched are required, and can be particularly preferably used, for example, as an electrode.

### REFERENCE SIGNS LIST

1a, 1b, 1c Layer body (MₘXₙ layer)

3a, 5a, 3b, 5b, 3c, 5c Modifier or terminal T

7a, 7b, 7c MXene layer

10 MXene (layered material)

11 Metal substrate

13 Conductive film

20 Conductive composite structure

1. A conductive composite structure comprising:
a metal substrate;
a conductive film on a surface of the metal substrate,
the conductive film comprising a layered material comprising one or plural layers, the one or plural layers comprising
a layer body represented by:
MₘXₙ
wherein M is at least one metal of Group 3, 4, 5, 6, or 7, X is a carbon atom, a nitrogen atom, or a combination thereof, n is not less than 1 and not more than 4, and m is more than n but not more than 5, and
a modifier or terminal T existing on a surface of the layer body, wherein T is a hydroxyl group, an oxygen atom, or a combination thereof; and
a residue derived from an organic compound having a hydroxyl group, a carbonyl group, or a combination thereof and having not less than 2 and not more than 8 carbon atoms bonded to each of the surface of the metal substrate and a surface of the layer body.

2. The conductive composite structure according to claim 1, wherein the organic compound contains at least one selected from the group consisting of isopropyl alcohol, N-methylpyrrolidone, and methyl ethyl ketone.

3. The conductive composite structure according to claim 1, wherein the metal substrate has a sheet form.

4. The conductive composite structure claim 1, wherein the metal substrate is an aluminum substrate, a copper substrate, or a stainless steel substrate.

5. The conductive composite structure claim 1, wherein M is at least one selected from the group consisting of Ti, Zr, Hf, V, Nb, Ta, Cr, and Mo.

6. The conductive composite structure claim 1, wherein the residue derived from the organic compound having the hydroxyl group, the carbonyl group, or the combination thereof has not less than 3 and not more than 7 carbon atoms.

7. The conductive composite structure according to claim 1, wherein the conductive composite structure satisfies the following (1) and (2):
(1) in a shaft center winding test, when the conductive composite structure is wound two turns around an aluminum round bar having a diameter of 4 mm, neither a crack in the conductive film nor a peeling-off of the conductive film from the metal substrate occurs; and
(2) in a tape peeling test, when a cellophane adhesive tape is attached to an area of an upper surface of the conductive film of the conductive composite structure, and thereafter the tape is peeled off, a total area that the conductive film is peeled off by the tape is not more than 20% of the area where the tape is attached to the conductive film.

8. An electrode comprising the conductive composite structure according to claim 1.

9. A method for producing a conductive composite structure, the method comprising:
(a) preparing a dispersion liquid in which a layered material is dispersed in a liquid medium comprising an organic compound having a hydroxyl group, a carbonyl group, or a combination thereof and having not less than 2 and not more than 8 carbon atoms, the layered material comprising one or plural layers, the one or plural layers comprising a layer body represented by:
MₘXₙ
wherein M is at least one metal of Group 3, 4, 5, 6, or 7, X is a carbon atom, a nitrogen atom, or a combination thereof, n is not less than 1 and not more than 4, and m is more than n but not more than 5, and
a modifier or terminal T existing on a surface of the layer body, wherein T is a hydroxyl group, an oxygen atom, or a combination thereof;
(b) applying the dispersion liquid to a surface of a metal substrate; and
(c) subjecting the metal substrate to which the dispersion liquid has been applied to a heat treatment so as to form the conductive composite structure comprising the metal substrate and a conductive film on the surface of the metal substrate.

10. The production method according to claim 9, wherein the heat treatment is performed at a temperature not lower than 70° C. and not higher than 200° C.

11. The production method according to claim 9, wherein the organic compound contains at least one selected from the group consisting of isopropyl alcohol, N-methylpyrrolidone, and methyl ethyl ketone.

12. The production method according to claim 9, wherein the metal substrate has a sheet form.

13. The production method according to claim 9, wherein the metal substrate is an aluminum substrate, a copper substrate, or a stainless steel substrate.

14. The production method according to claim 9, wherein M is at least one selected from the group consisting of Ti, Zr, Hf, V, Nb, Ta, Cr, and Mo.

15. The production method according to claim 9, wherein the organic compound having the hydroxyl group, the carbonyl group, or the combination thereof has not less than 3 and not more than 7 carbon atoms.

16. The production method according to claim 9, wherein a content ratio of the layered material in the dispersion liquid is 20% to 95% by mass.

17. The production method according to claim 9, wherein the produced conductive composite structure satisfies the following (1) and (2):
(1) in a shaft center winding test, when the conductive composite structure is wound two turns around an aluminum round bar having a diameter of 4 mm, neither a crack in the conductive film nor a peeling-off of the conductive film from the metal substrate occurs;
(2) in a tape peeling test, when a cellophane adhesive tape is attached to an area of an upper surface of the conductive film of the conductive composite structure, and thereafter the tape is peeled off, a total area that the conductive film is peeled off by the tape is not more than 20% of the area where the tape is attached to the conductive film.
